(19) United States
(12) Patent Application Publication
(10) Pub. No.: US 2025/0186097 A1
(43) Pub. Date: Jun. 12, 2025

(54) SYSTEMS AND METHODS FOR PULSED FIELD ABLATION WITH INCREASED PULSE PERIODS

(71) Applicant: St. Jude Medical, Cardiology Division, Inc., St. Paul, MN (US)

(72) Inventors: Derek Sutermeister, Ham Lake, MN (US); Isaac Remer, Richfield, MN (US)

(21) Appl. No.: 18/973,952

(22) Filed: Dec. 9, 2024

Related U.S. Application Data
(60) Provisional application No. 63/608,568, filed on Dec. 11, 2023.

(51) Int. Cl. A61B 18/00

(52) U.S. Cl. CPC A61B 18/00; A61B 2018/0016; A61B 2018/00613; A61B 2018/00642; A61B 2018/0075; A61B 2018/00761; A61B 2018/00767; A61B 2018/00827

(57) ABSTRACT

An electroporation system is provided. The electroporation system includes a catheter comprising a plurality of electrodes, and a pulse generator coupled to the catheter, the pulse generator configured to generate a waveform to be delivered using at least one of the plurality of electrodes. The waveform includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

### CROSS REFERENCE TO RELATED APPLICATIONS

This application claims priority to U.S. Provisional Patent Application No. 63/608,568 filed on Dec. 11, 2023, which is incorporated by reference herein in its entirety.

### FIELD OF THE DISCLOSURE

The present disclosure relates generally to tissue ablation systems. In particular, the present disclosure relates to systems and methods for pulsed field ablation that use increased pulse periods.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as ventricular and atrial arrhythmias (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a substantially non-thermal ablation technique that involves applying strong electric-fields that induce pore formation in the cellular membrane. The electric field may be induced by applying a relatively short duration pulse which may last, for instance, from a nanosecond to several milliseconds and generate a moderate amount of heating. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, voltage pulses may range from less than about 50 volts to about 10,000 volts or higher. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to minimize thermal heating of the tissue, and to have little to no skeletal muscle recruitment (i.e., avoiding muscle contractions). In addition, it is also generally desirable to reduce the likelihood of waveforms generating sustained atrial arrhythmias.

### BRIEF SUMMARY OF THE DISCLOSURE

In one aspect, an electroporation system is provided. The electroporation system includes a catheter comprising a plurality of electrodes, and a pulse generator coupled to the catheter, the pulse generator configured to generate a waveform to be delivered using at least one of the plurality of electrodes. The waveform includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

In another aspect, a pulse generator for use with an electroporation system is provided. The pulse generator is configured to be coupled to a catheter including a plurality of electrodes and is configured to generate a waveform to be delivered using at least one of the plurality of electrodes. The waveform includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

In yet another aspect, a method for electroporation therapy is provided. The method includes generating, using a pulse generator, a waveform that includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms. The method further includes delivering the waveform using at least one of a plurality of electrodes on a catheter, wherein the pulse period of the waveform facilitates increasing lesion depth.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIGS. 2A and 2B are views of one embodiment of a catheter assembly that may be used with the system shown in FIG. 1.

FIGS. 3A-3C are views of alternative embodiments of a catheter assembly that may be used with the system shown in FIG. 1.

FIG. 4 is one embodiment of a waveform that may be delivered using the system shown in FIG. 1.

FIG. 5A is a graph showing changes in transmembrane potential over time during a waveform with a 1 ms pulse period.

FIG. 5B is a graph showing changes in transmembrane potential over time during a waveform with a 100 μs pulse period.

### DETAILED DESCRIPTION OF THE DISCLOSURE

The present disclosure provides systems and methods for electroporation. An electroporation system includes a catheter comprising a plurality of electrodes, and a pulse generator coupled to the catheter, the pulse generator configured to generate a waveform to be delivered using at least one of the plurality of electrodes. The waveform includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

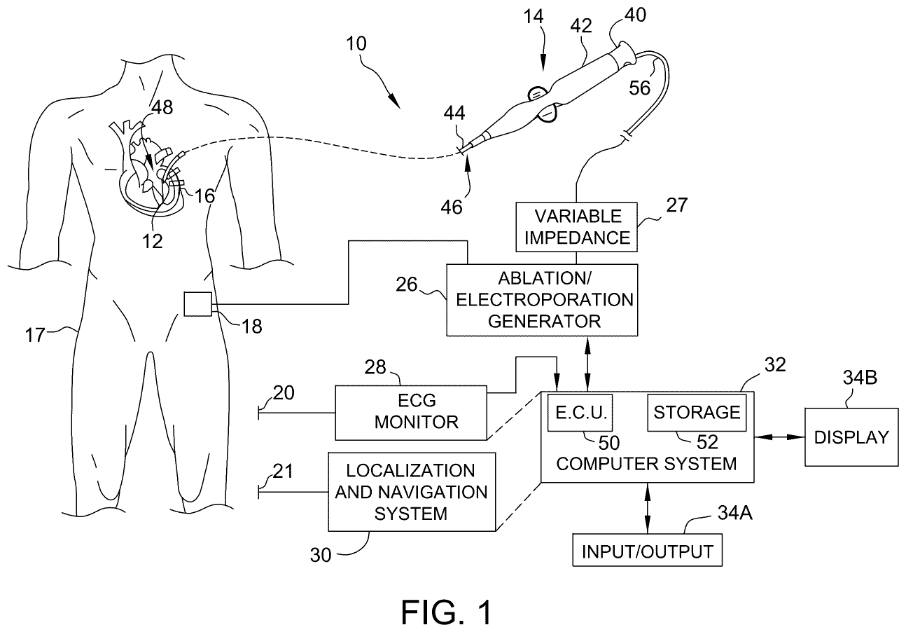

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

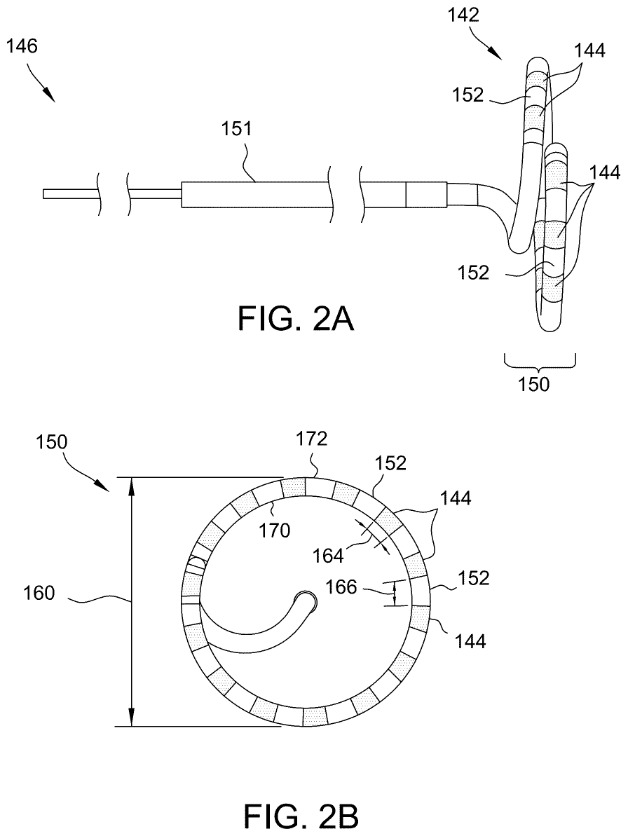

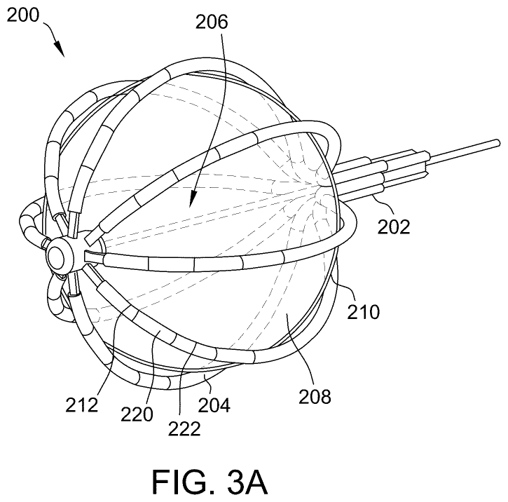

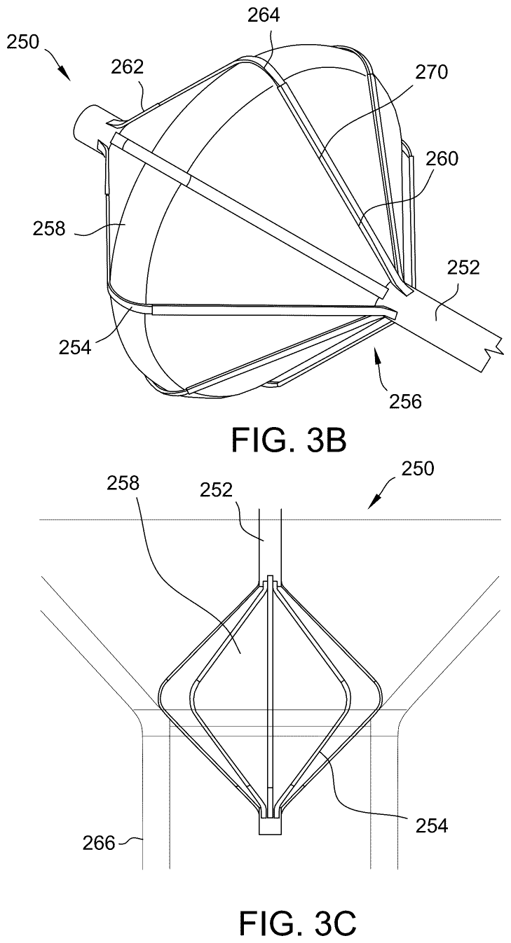

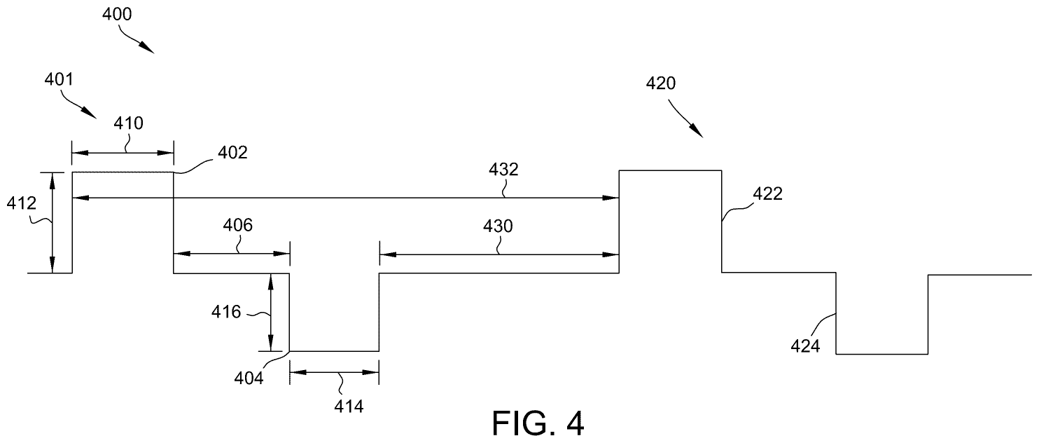

System 10 may be used for irreversible electroporation (IRE) to destroy tissue. In particular, system 10 may be used for electroporation-induced therapy that includes delivering electrical pulses in such a manner as to directly cause an irreversible loss of plasma membrane integrity leading to its breakdown and cell destruction. This mechanism of cell destruction may be viewed as an “outside-in” process, meaning that the disruption of the outside plasma membrane of the cell causes detrimental effects to the inside of the cell. Sometimes these electrical pulses may directly manipulate and damage the intracellular organelles to induce cell death, without causing a significant amount of damage to the cell membrane. Typically, for classical plasma membrane electroporation, electric energy may be delivered as a pulsed electric field in the form of short-duration pulses (e.g., having a 10 nanosecond (ns) to 100 millisecond (ms) duration) between closely spaced electrodes capable of delivering an electric field strength of about 0.05 to 100.0 kilovolts/centimeter (kV/cm). System 10 may be used for high output (e.g., high voltage and/or high current) electroporation procedures. Further, system 10 may be used with a loop catheter such as that depicted in FIGS. 2A and 2B, and/or with a basket catheter such as those depicted in FIGS. 3A-3C. In some embodiments, system 10 is used for reversible electroporation instead of or in addition to irreversible electroporation.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein. Further, irreversible electroporation may be used for focal ablation procedures. Notably, the embodiments described herein may be used with any suitable irreversible electroporation application.

It should be understood that while the energization strategies are described as involving square wave pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption (e.g., through pore formation and/or other cell damage) through application of a high-voltage electric field. Thus, electroporation may avoid some possible thermal effects that may occur when using radio frequency (RF) energy. This “colder therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). Further, in some embodiments, in a multiplexing arrangement, therapy may repeatedly switch between using a different return electrodes 18, 20, 21. In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric energy that is delivered via electrode assembly 12 as a pulsed electric field in the form of short-duration square wave pulses (e.g., a nanosecond to several milliseconds duration, or any duration suitable for electroporation) between closely spaced electrodes capable of delivering an electric field strength (i.e., at the tissue site) of about 0.05 to 100.0 kV/cm. The amplitude and pulse width needed for irreversible electroporation are inversely related. That is, as pulse widths are decreased, the amplitude may generally be increased to achieve chronaxie. The electric energy may be delivered, for example, using a fixed voltage delivery system (in which a fixed voltage is applied, independent of a patient impedance) or using a fixed current delivery system (in which a fixed current is achieved by adjusting the voltage based on the patient impedance). In a fixed current delivery system, the patient impedance may be determined, for example, by delivering a relatively small voltage pulse and measuring current to calculate impedance, or by delivering an AC current waveform and measuring voltage to calculate impedance. A fixed current system may also involve measuring current (e.g., before or during therapy delivery) and adjusting voltage accordingly. For example, current may be measured during delivery of a first therapy pulse (or during a pre-therapy pulse with a relatively low voltage), an impedance may be calculated from the measured current, and the voltage may be adjusted (and then left unchanged) to obtain the desired current during therapy. In another example, current may be measured during one or more pulses delivered during therapy, the impedance may be calculated for each pulse that the current was measured for, and the voltage of each subsequent pulse may be actively adjusted.

Electroporation generator 26, sometimes also referred to herein as a DC energy source, is a biphasic electroporation generator 26 configured to generate a series of energy pulses that all produce current in two directions (i.e., positive and negative pulses). In other embodiments, electroporation generator is a monophasic or polyphasic electroporation generator. In some embodiments, electroporation generator 26 is configured to output energy in pulses at selectable energy levels, such as fifty joules, one hundred joules, two hundred joules, and the like. Other embodiments may have more, or fewer energy settings and the values of the available setting may be the same or different (settings may include, e.g., waveform parameters, voltage, current, number of applications, etc.). For successful electroporation, some embodiments utilize the two hundred joule output level. For example, electroporation generator 26 may output a pulse having a peak magnitude from about 10 Volts (V) to about 20,000 V. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N. V., the AURORA® system of Northern Digital Inc., commonly available fluoroscopy systems, or a magnetic location system such as the gMPS system from Mediguide Ltd

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation and cell death, may be implemented using the systems and methods described herein. In some cases, PFA may be used at specific cardiac tissue sites such as the pulmonary veins to perform a pulmonary vein isolation (PVI), or to perform focal ablation. In PFA, electric fields may be applied between adjacent electrodes (in a bipolar approach) or between one or more electrodes and a return patch (in a monopolar approach). There are advantages and disadvantages to each of these approaches.

Both approaches, using an appropriate electrode geometry, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions (depending on, for example, tissue thickness). To monitor operation of system 10, one or more impedances between catheter electrodes 144 and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

FIGS. 2A and 2B are views of one embodiment of a catheter assembly 146 that may be used with catheter 14 in system 10. Catheter assembly 146 may be referred to as a loop catheter.

Those of skill in the art will appreciate that, in other embodiments, any suitable catheter may be used. That is, the systems and methods described herein are not limited to use with the particular catheter assemblies shown. For example, the systems and methods described herein may be implemented in a linear catheter, a grid catheter (e.g., a catheter including a number of splines arranged in a plane, each spline including one or more electrodes), and/or a focal ablation catheter (e.g., such as the Abbott TactiFlex catheter and TactiCath catheter).

Specifically, FIG. 2A is a side view of catheter assembly 146 with a variable diameter loop 150 at a distal end 142. FIG. 2B is an end view of variable diameter loop 150 of catheter assembly 146. Those of skill in the art will appreciate that the methods and systems described herein may be implemented using any suitable catheter (e.g., fixed loop catheters, linear catheters, basket catheter, etc.). As shown in FIGS. 2A and 2B, variable diameter loop 150 is coupled to a distal section 151 of shaft 44.

Variable diameter loop 150 is selectively transitionable between an expanded (also referred to as “open”) diameter 160 (shown in FIG. 2A) and a retracted (also referred to as “closed”) diameter 160 (not shown). In the example embodiment, an expanded diameter 160 is twenty eight mm and a retracted diameter 160 is fifteen mm. In other embodiments, diameter 160 may be variable between any suitable open and closed diameters 160.

In the embodiment shown, variable diameter loop 150 includes fourteen catheter electrodes 144 substantially evenly spaced around the circumference of variable diameter loop 150 in the expanded configuration. In the retracted configuration, one or more of electrodes 144 may overlap. In other embodiments, other arrangements of catheter electrodes 144 may be implemented. For example, in one embodiment, variable diameter loop 150 includes twelve catheter electrodes 144.

Catheter electrodes 144 are platinum ring electrodes configured to conduct and/or discharge electrical current in the range of one thousand volts and/or ten amperes. In other embodiments, variable diameter loop 150 may include any suitable number of catheter electrodes 144 made of any suitable material. Catheter electrodes 144 may include any catheter electrode suitable to conduct high voltage and/or high current (e.g., in the range of one thousand volts and/or ten amperes). Each catheter electrode 144 is separated from each other catheter electrode by an insulated gap 152. In the example embodiment, each catheter electrode 144 has a same length 164 (shown in FIG. 2B) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter loop 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter loop 150, while still allowing enough flexibility to allow variable diameter loop 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter loop 150 while also decreasing current density (by increasing the surface area) on electrodes 144, which may help prevent arcing and reduce thermal effects during electroporation operations. Increasing length 164 too much, however, may prevent variable diameter loop 150 from forming a smooth circular shape and may limit the closed diameter 160 of variable diameter loop 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing and heating during electroporation, and may result in larger additional system resistances needing to be added to prevent arcing. Moreover, in order to get a desired, even coverage about the circumference of variable diameter loop 150, more catheter electrodes 144 may be needed if length 164 is decreased. Increasing the number of catheter electrodes 144 on variable diameter loop 150 may prevent variable diameter loop 150 from being able to be contracted to a desired minimum diameter 160.

FIG. 3A is a perspective view of an alternative catheter assembly 200 that may be used with catheter 14. Catheter assembly 200 may be referred to as a basket catheter. Catheter assembly 200 includes a shaft 202 and a plurality of splines 204 surrounding a distal portion 206 of shaft 202. In this embodiment, catheter assembly 200 also includes a balloon 208 enclosed by splines 204. Balloon 208 may be selectively inflated to fill the space between splines 204. Notably, balloon 208 functions as an insulator, and generally reduces energy losses, which may result in increased lesion size. In some embodiments, balloon 208 may be filled with a cold media (e.g., a cryofluid or cold saline). Further, in some embodiments balloon 208 may be a double-layered balloon.

Each spline 204 includes a proximal end 210 coupled to shaft 202 and a distal end 212 coupled to shaft 202. From proximal end 210 to distal end 212, spline 204 has an arcuate shape that extends radially outward.

In this embodiment, each spline 204 includes a plurality of individual electrodes 220. For example, each spline 204 may include an elastic material (e.g., Nitinol) covered in a polymer tube 222, with individual electrodes 220 attached to an exterior of polymer tube 222. In the embodiment shown, each spline 204 includes two electrodes 220. Further, as shown in FIG. 2, electrodes 220 are generally positioned closer to distal end 212 than proximal end 210 to correspond to portions of spline 204 that will contact the pulmonary vein.

Alternatively, each spline 204 may include any suitable number and arrangement of electrodes 220. For example, in some embodiments, each spline 204 includes four electrodes 220.

In this embodiment, alternating splines 204 alternate polarities. That is, electrodes 220 on a particular spline 204 have the same polarity, but electrodes 220 on a particular spline 204 have a different polarity than electrodes 220 on adjacent splines 204. Alternatively, any suitable polarization scheme may be used. During delivery, splines 204 may be collapsed in towards shaft 202. Subsequently, to perform ablation, splines 204 are deployed to extend radially outward.

Splines 204 may all have the same length, or at least some of splines 204 may have different lengths. Further, insulating material on each spline 204 may have the same length, or at least some splines 204 may have insulating material with different lengths. In addition, in some embodiments, catheter assembly 200 includes a distal electrode (not shown) positioned distal of splines 204. The distal electrode may be used to perform point ablation (e.g., by creating a bipole between the distal electrode and one of splines 204), and/or may be used for visualization/mapping purposes (e.g., using the distal electrode in combination with an electrode on shaft 202).

FIG. 3B is a perspective view of an alternative catheter assembly 250 that may be used with catheter 14, and FIG. 3C is a side schematic view of catheter assembly 250. Like catheter assembly 200 (shown in FIG. 3A), catheter assembly 250 may be referred to as a basket assembly.

Catheter assembly 250 includes a shaft 252 and a plurality of splines 254 surrounding a distal portion 256 of shaft 252. In this embodiment, catheter assembly 250 includes a balloon 258 enclosed by splines 254. Balloon 258 may be selectively inflated to occupy the space between splines 254. Notably, balloon 258 functions as an insulator, and generally reduces energy, which may result in increased lesion size.

Each spline 254 includes a proximal end 260 coupled to shaft 252 and a distal end 262 coupled to shaft 252. From proximal end 260, spline 1004 extends radially outward to an inflection point 264, and then extends radially inward to distal end 262. FIG. 3C shows catheter assembly 250 positioned within the pulmonary vein 266.

A body of each spline 254 is made of an elastic material (e.g., Nitinol), and functions as a relatively large electrode. In this embodiment, alternating splines 254 alternate polarities. That is, each positive spline 254 is positioned between two negative splines 254 and vice-versa. Alternatively, any suitable polarization scheme may be used.

To control the ablation zone of each spline 254, portions of each spline 254 may be covered with insulating material 270 (e.g., heat-shrink or polymer tubing or spray or dip coat with polyimide or PEBAX), and the exposed portions of splines 254 function as electrodes. In the embodiment shown in FIGS. 3B and 3C, inflection point 264 and portions of spline 254 between inflection point 264 and distal end 262 are generally exposed, while portions of spline 254 between inflection point 264 and proximal end 260 are generally insulated. This results in the portions of spline 254 that contact pulmonary vein 266 being exposed (see FIG. 3C). Alternatively, any suitable insulation configuration may be used.

During delivery, splines 254 and balloon 258 may be collapsed. To perform ablation, splines 254 are deployed with inflection points 264 extending radially outward, and balloon 258 is selectively inflated to occupy the space between splines 254.

The combination of balloon 258 and splines 254 facilitates straightforward delivery and deployment of catheter assembly 250. Further, balloon 258 drives more energy into ablated tissue, and stabilizes splines 254 to prevent lateral movement. In addition, using splines 254 as electrodes instead of individual smaller electrodes may facilitate reducing the cost and increasing the reliability of catheter assembly 250.

Splines 254 may all have the same length, or at least some of splines 254 may have different lengths. Further, insulating material 270 on each spline 254 may have the same length, or at least some splines 254 may have insulating material 270 with different lengths. In addition, in some embodiments, catheter assembly 250 includes a distal electrode (not shown) positioned distal of splines 254. The distal electrode may be used to perform point ablation (e.g., by creating a bipole between the distal electrode and one of splines 254), and/or may be used for visualization/mapping purposes (e.g., using the distal electrode in combination with an electrode on shaft 252).

Those of skill the art will appreciate that catheter assembly 146 (shown in FIG. 2A and 2B), catheter assembly 200 (shown in FIG. 3A), and catheter assembly 250 (shown in FIGS. 3B and 3C) are merely examples. Notably, the systems and methods described herein may be implemented using any suitable catheter assembly.

For electroporation therapy, waveforms are generated using a pulse generator (e.g., electroporation generator 26 (shown in FIG. 1)) and applied between two or more catheter electrodes (i.e., a bipolar approach) or between individual catheter electrodes and a return patch (i.e., a monopolar approach). The waveforms may be monophasic, biphasic (i.e., having both a positive pulse and a negative pulse), or polyphasic. Further, the waveforms may include one or more bursts of pulses (with each burst including multiple pulses). Further, the waveforms are defined by multiple parameters (e.g., pulse width, pulse amplitude, frequency, etc.).

Different waveforms may be used to achieve different goals. For example, some waveforms may result in larger or smaller lesion size than other waveforms. Further, some waveforms result or higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions).

FIG. 4 is one embodiment of a waveform 400 that may be delivered, for example, using system 10 (shown in FIG. 1). Waveform 400 includes a first pulse pattern 401 that includes a positive pulse 402 followed by a negative pulse 404. Further, there is an intrapulse delay 406 between the positive and negative pulses 402 and 404.

As shown in FIG. 4, positive pulse 402 has a first pulse width 410 and a first pulse amplitude 412. Similarly, negative pulse 404 has a second pulse width 414 and a second pulse amplitude 416. First pulse pattern 401 may be symmetric (i.e., with first pulse width 410 and first pulse amplitude 412 substantially equal to second pulse width 414 and second pulse amplitude 416) or asymmetric (i.e., with at least one of first pulse width 410 and first pulse amplitude 412 different from second pulse width 414 and second pulse amplitude 416).

When first pulse amplitude 412 and second pulse amplitude 416 are both non-zero, first pulse pattern 401 is biphasic (i.e., as shown in FIG. 4). For a monophasic waveform, one of first pulse amplitude 412 and second pulse amplitude 416 is zero. For example, if first pulse amplitude 412 is zero, first pulse pattern 401 is monophasic with single negative pulse 404. If second pulse amplitude 416 is zero, first pulse pattern 401 is monophasic with single positive pulse 402.

In one biphasic example, first and second pulse widths 410 and 414 may each be 3 microseconds (3 μs), with an intrapulse delay of 1 μs. This may be referred to as a 3-1-3 waveform (i.e., first pulse width of 3 μs—intrapulse delay of 1 μs—second pulse width of 3 μs). First and second pulse amplitudes 412 and 416 may each be, for example, on the order of 1800 Volts (1800V).

In one monophasic example, first pulse width 410 is 0 μs, and second pulse width 414 is 3 μs, with an intrapulse delay of 1 μs. This may be referred to as a 0-1-3 waveform (i.e., first pulse width of 0 μs—intrapulse delay of 1 μs—second pulse width of 3 μs). Second pulse amplitude 416 may be, for example, on the order of 1800V. In another example, the intrapulse delay may be 0 μs.

In this embodiment, waveform 400 further includes a second pulse pattern 420 that follows first pulse pattern 401. Second pulse pattern 420 may be substantially similar to first pulse pattern 401 (e.g., second pulse pattern 420 may be a biphasic pulse pair with a positive pulse 422 followed by a negative pulse 424). Alternatively, second pulse pattern 420 may have different parameters from first pulse pattern 401 (e.g., a different number of pulses, different pulse amplitudes, different pulse lengths, and/or difference pulse shapes).

An interpulse delay 430 is defined between the end of first pulse pattern 401 (e.g., the end of negative pulse 404 of first pulse pattern 401) and the beginning of second pulse pattern 420 (e.g., the beginning of positive pulse 422 of second pulse pattern 420). Further, a pulse period 432 is defined between the beginning of first pulse pattern 401 (e.g., the beginning of positive pulse 402 of first pulse pattern 401) and the beginning of second pulse pattern 420 (e.g., the beginning of positive pulse 422 of second pulse pattern 420).

Notably, it has been observed that the length of a pulse period (such as pulse period 432) may significantly impact both lesion formation and patient movement. Specifically, increased pulse periods may improve lesion formation (e.g., by increasing lesion depth). This should be balanced with maintaining tip stability and maintaining low or moderate levels of patient movement. For example, pulse periods greater than 100 μs or greater than 200 μs (e.g., pulse periods in a range from 100 μs to 1 millisecond (ms)) may generally improve lesion formation. As additional examples, in some embodiments, a pulse period in a range from 1 ms to 100 ms, or more particularly in a range from 1 ms to 3.5 ms, or more particularly in a range from 3.5 ms to 100 ms, or more particularly in a range from 5 ms to 100 ms, or even more particularly in a range from 10 ms to 100 ms may generally improve lesion formation.

The impact of increased pulse periods has been demonstrated for both monophasic and biphasic waveforms. Specifically, it was experimentally verified that, at pulse periods below 5 ms, lesion formation drops significantly (with rapid improvements to lesion depth at pulse periods greater than 1 ms). In contrast, relatively consistent, improved lesion formation was demonstrated for pulse periods at or above 5 ms.

Increased pulse periods may, in some scenarios, reduce patient movement (e.g., due to skeletal muscle recruitment (SMR)). This is important for catheter stability, patient comfort, physician perception, and treatment efficiency, and is particularly important for monopolar approaches and bipolar approaches with a substantial distance between the bipolar electrodes.

Longer pulse periods result in less pulses over a given period of time, which facilitates reducing microbubble formation and reducing heat generation, while maintaining or increasing lesion depth.

Longer pulse periods also result in reduced dielectric stress on catheter insulation (as longer pulse periods allows for increased charge decay), simplifying catheter insulation constraints and also enabling delivery of higher therapy voltages and currents and further enhancing lesion generation. Longer pulse periods also allow for additional cooling between pulses, reducing thermal profiles of the electroporation therapy.

In the embodiment of FIG. 4, first pulse pattern 401 and second pulse pattern 420 are delivered between the same set of electrodes. That is, first pulse pattern 401 is delivered between one or more first electrodes and one or more second electrodes (e.g., in a monopolar, bipolar, or multipolar approach), and second pulse pattern 420 is delivered between the same one or more first electrodes and one or more second electrodes. In this embodiment, although first pulse pattern 401 and second pulse pattern 420 are delivered consecutively relative to the one or more first electrodes and the one or more second electrodes, additional pulses may be delivered between different electrodes (e.g., between one or more third electrodes and one or more fourth electrodes) in the interim between first pulse pattern 401 and second pulse pattern 420.

During an electrode channel window, one or more pulses (e.g., monophasic and/or biphasic waveforms) are delivered by selected electrodes. For example, waveform 400 is an example set of pulses delivered between particular electrodes. Those of skill in the art will appreciate that many different electrode energization schemes are possible.

For example, consider catheter assembly 250 (shown in FIGS. 3B and 3C), which includes eight electrodes (with each spline 254 functioning as an electrode). The eight electrodes may be numbered, in order around a circumference of catheter assembly 250, E1, E2, . . . . E8. In one example energization scheme, during a first electrode channel window, a first waveform is delivered between E1 (set to a positive voltage) and E2 (set to a negative voltage), and a second waveform is simultaneously delivered between E5 (set to a positive voltage) and E6 (set to a negative voltage). During a second electrode channel window, a first waveform is delivered between E3 (set to a positive voltage) and E4 (set to a negative voltage), and a second waveform is simultaneously delivered between E7 (set to a positive voltage) and E8 (set to a negative voltage). Again, those of skill in the art will appreciate that many different electrode energization schemes are possible.

Multiple electrode channel windows constitute a loop. That loop completes when the pattern of electrode energization begins repeating. For example, a single loop may include the first and second electrode channel windows described above. Once that loop completes, a subsequent loop would begin, starting again with the first electrode channel window. Multiple loops constitute a burst, and a therapy session may include one or more bursts. In general, the period of time between subsequent bursts is longer than the period of time between loops within a burst. That is, i) the time between the end of a last pulse of a last loop in a first burst and the beginning of a first pulse of a first loop in a second, subsequent burst is greater than ii) the time between the end of a last pulse of the second last loop in the first burst and the beginning of a first pulse of the last loop in the first burst.

Notably, as pulse periods increase (e.g., above 5 ms, as described above), the number of pulse patterns able to be delivered within a given time period is reduced. For example, an 80 ms R-wave gating window may correspond to a therapeutic delivery window of 69 ms (due to switching and response times) for the burst. This may allow for one to nine pulse patterns per burst (e.g., a single pulse pattern, or two pulse patterns with a pulse period of approximately 69 ms, or three pulse patterns with a pulse period of approximately 34.5 ms, or four pulse patterns with a pulse period of approximately 23 ms, etc., up to nine pulse patterns with a pulse period of approximately 8.625 ms). In some embodiments, the pulse period could vary between pulse patterns, resulting in uneven spacing of the pulse patterns. Alternatively, the pulse patterns are evenly spaced within the burst.

Notably, for longer burst periods, a higher number of pulses per burst may be included while still maintaining increased pulse periods. For example, for a burst period of approximately 150 ms, one hundred and fifty pulse patterns could be included within the burst at a pulse period of 1 ms. Those of skill in the art will appreciate that, depending on the length of the burst period, any suitable number of pulses may be included using the pulse periods disclosed herein.

Those of skill in the art will also appreciate that lower pulse periods generally allow for additional pulses per burst. For example, at a pulse period of 1 ms, seventy pulses may be delivered within a burst (e.g., at a burst period of approximately 69 ms). At a pulse period of 100 μs, seven hundred pulses having a relatively small pulse width (e.g., on the order of nanoseconds) may be delivered within a burst (e.g., at a burst period of approximately 69 ms).

In general, it has been demonstrated that, for a relatively small number of total overall pulse patterns, spreading pulse patterns out over multiple bursts (i.e., with a larger pulse period) is more effective than squeezing multiple pulse patterns into fewer bursts (i.e., with a smaller pulse period).

As explained above, it has been demonstrated that longer pulse periods facilitate improving lesion formation. For example, in one experiment, for a 3-1-3 waveform, a pulse period of 8 ms resulted in significant improvements to lesion depth relative to a pulse period of 330 μs. Similarly, in another experiment, for a 2-1-2 waveform, a pulse period of 5 ms resulted in significant improvements to lesion depth relative to a pulse period of 100 μs. Those of skill in the art will appreciate that other waveforms (e.g., a 5-1-5 waveform, a 1-1-1-waveform, etc.) should result in similar improvements.

The following provides a brief explanation why longer pulse periods may result in improved lesion depth. As will be understood by those of skill in the art, in electroporation applications, when cells are subjected to relatively short duration, high voltage electrical pulses, a transmembrane potential of the cells increases. When the transmembrane potential increases beyond a certain electroporation threshold (e.g., 0.5V), for a relatively thin membrane (e.g., ˜5 nanometers (ms)), there is a several-fold enhancement of the electrical field (e.g., 100 MV/cm), causing pore formation. This phenomenon is electroporation, and the pores formed can either reseal (reversible electroporation) or permanently remain open (irreversible electroporation). In addition to electric field strength, the duration of electric field exposure also determines the outcome of electroporation.

Once the transmembrane potential increases beyond the electroporation threshold, it must stay above the threshold for a certain amount of time to cause electroporation. Longer pulse widths, higher voltages, and increased numbers of pulses all facilitate keeping the transmembrane potential above the electroporation threshold.

For monophasic pulses, the membrane discharges naturally, allowing the transmembrane potential to be above the electroporation threshold for a relatively long time period, causing an effective electroporation. Depending upon the time between pulses (i.e., the pulse period) it is possible to generate waveforms that facilitate maximizing time above the electroporation threshold, while still applying relatively few pulses. Pulse length for such waveforms may also be controlled to facilitate maximizing time above the threshold (e.g., a nanosecond pulse will increase the transmembrane potential less than a microsecond pulse).

In contrast, for biphasic pulses, there is a reversal of the electric field when switching between pulses of different polarity. This reversal discharges the membrane (or charges the membrane to the opposite polarity), in a phenomenon referred to as “assistive discharge”. Assistive discharge reduces the amount of time that the transmembrane potential remains above the electroporation threshold. In general, the opposite polarity phases of biphasic pulses work against one another by charging the membrane in opposite directions. In an asymmetric biphasic pulse pair, a stronger second phase generally results in more effective electroporation.

When multiple biphasic pulse pairs are applied, subsequent pulse pairs introduce assisted discharge on the previous pulse pairs. For example, consider a waveform including a first biphasic pulse pair (with a first positive pulse followed by a first negative pulse) followed by a second biphasic pulse pair (with a second positive pulse followed by a second negative pulse). FIG. 4, discussed above, shows one example of such a waveform. In this waveform, the second positive pulse will result in assistive discharge of the charge resulting from the first negative pulse, reducing the time above the electroporation threshold, and resulting in less effective electroporation (e.g., shallower lesions).

The phenomenon can be reduced by increasing the pulse period (e.g., increasing the time between the end of the first negative pulse and the beginning of the second negative pulse in the above example). Therefore, increasing the pulse period increases the time that the transmembrane potential remains above the electroporation threshold, resulting in more effective electroporation (e.g., deeper lesions). Accordingly, at shorter periods, additional pulses (or higher voltage and/or pulse width) would be required to achieve the same electroporation efficiency as at longer pulse periods.

Cell size, tissue type, and conductivity all affect the natural discharge time for the membrane (and the amount of electroporation realized). Accordingly, different pulse periods may be used for different cells and tissue types. Further, different pulse periods may be used for different applied voltages and pulse widths. In some embodiments, conductivity of the cellular microenvironment could be manipulated (e.g., using irrigation) to facilitate controlling lesion depth.

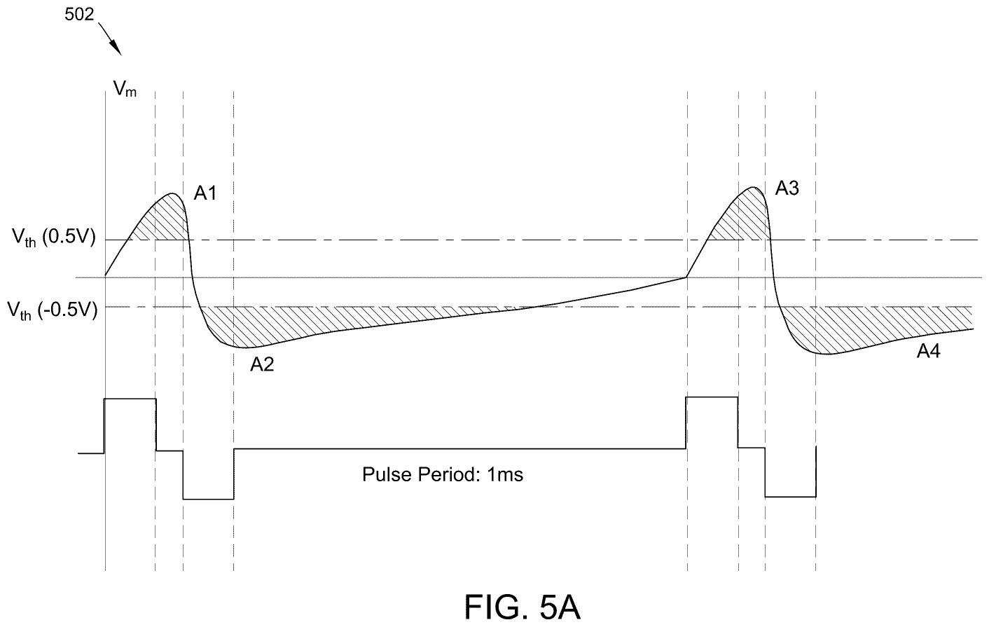

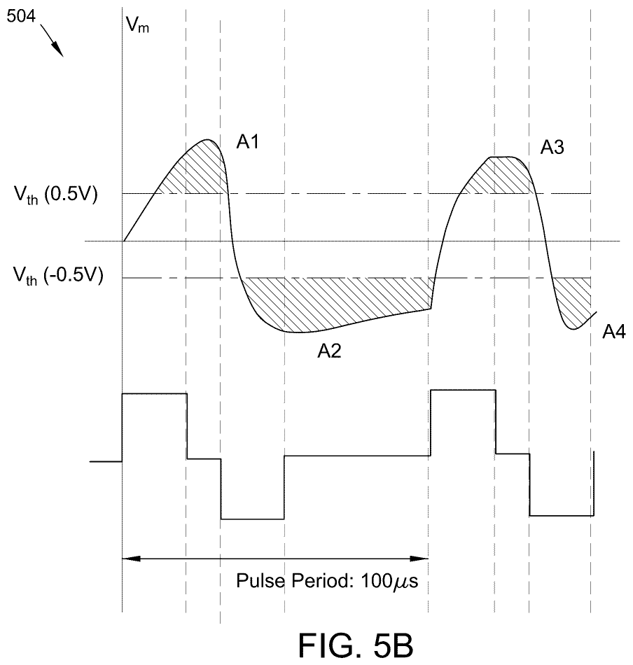

FIG. 5A is a graph 502 showing changes in the transmembrane potential over time during a waveform with a 1 ms pulse period. In contrast, FIG. 5B is a graph 504 showing changes in the transmembrane potential over time during a waveform with a 100 μs pulse period. As can be seen from FIG. 5B, the shorter pulse period results in the assisted discharge causing an abrupt cut off of the natural decay of the voltage, reducing lesion depth. In contrast, as can be seen from FIG. 5A, the longer pulse period allows the voltage to decay naturally.

For similar reasons, the last pulse in a burst may be the most effective, as it will be a relatively long time until the next pulse begins. Accordingly, in some embodiments, the last pulse in a burst (either both polarity phases, or just the second polarity phase) has a higher voltage and/or longer pulse width than previous pulses in the burst.

As noted above, in addition to improving lesion depth, increased pulse periods may also facilitate reducing SMR and phrenic stimulation, both of which impact patient movement. For electrode configurations with relatively close proximity, the impact of increased pulse periods may be relatively small. However, for electrodes that are spaced relatively far from one another, increases in pulse period have been shown to reduce patient movement.

Similarly, as noted above, spreading a given number of pulses over more bursts generally yields a better lesion depth than delivering the same number of pulses in fewer bursts. For example, it has been demonstrated that delivering two therapy applications, each application including ten loops of pulses within a single burst resulted in shallower lesion depth that delivering two therapy applications that each included ten bursts of pulses with a single loop in each burst.

The systems and methods described herein are directed to electroporation waveforms. An electroporation system includes a catheter comprising a plurality of electrodes, and a pulse generator coupled to the catheter, the pulse generator configured to generate a waveform to be delivered using at least one of the plurality of electrodes. The waveform includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An electroporation system comprising:
a catheter comprising a plurality of electrodes; and
a pulse generator coupled to the catheter, the pulse generator configured to generate a waveform to be delivered using at least one of the plurality of electrodes, the waveform including:
a first pulse pattern: and
a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

2. The electroporation system in accordance with claim 1, wherein the pulse period is in a range from 5 ms to 100 ms.

3. The electroporation system in accordance with claim 1, wherein the pulse period is in a range from 10 ms to 100 ms.

4. The electroporation system in accordance with claim 1, wherein the first and second pulse patterns are included within a burst, and wherein the burst includes one hundred fifty total pulse patterns or less.

5. The electroporation system in accordance with claim 1, wherein the second pulse pattern is the last pulse pattern in a burst, and wherein the second pulse pattern has a larger applied voltage and/or a longer pulse width than the first pulse pattern.

6. The electroporation system in accordance with claim 1, wherein the first and second pulse patterns are delivered between at least one first electrode and at least one second electrode, and wherein, between delivery of the first and second pulse patterns, at least one additional pulse is delivered between at least one third electrode and at least one fourth electrode.

7. The electroporation system in accordance with claim 1, wherein the electroporation system comprises a fixed current delivery system that is configured to i) measure current during delivery of at least one pulse and ii) adjust at least one subsequent pulse voltage based on the measured current.

8. A pulse generator for use with an electroporation system, the pulse generator configured to be coupled to a catheter including a plurality of electrodes and configured to generate a waveform to be delivered using at least one of the plurality of electrodes, the waveform including:
a first pulse pattern: and
a second pulse pattern that is consecutive to the first pulse pattern, wherein a pulse period defined between a start of the first pulse pattern and a start of the second pulse pattern is in a range from 1 millisecond (ms) to 100 ms, such that the waveform facilitates increasing lesion depth.

9. The pulse generator in accordance with claim 8, wherein the pulse period is in a range from 5 ms to 100 ms.

10. The pulse generator in accordance with claim 8, wherein the pulse period is in a range from 10 ms to 100 ms.

11. The pulse generator in accordance with claim 8, wherein the first and second pulse patterns are included within a burst, and wherein the burst includes one hundred fifty total pulse patterns or less.

12. The pulse generator in accordance with claim 8, wherein the second pulse pattern is the last pulse pattern in a burst, and wherein the second pulse pattern has a larger applied voltage and/or a longer pulse width than the first pulse pattern.

13. The pulse generator in accordance with claim 8, wherein at least one of the first and second pulse patterns is a biphasic pulse pattern.

14. The pulse generator in accordance with claim 8, wherein at least one of the first and second pulse patterns is a monophasic pulse pattern.

15. A method for electroporation therapy, the method comprising:
generating, using a pulse generator, a waveform that includes a first pulse pattern, and a second pulse pattern that is consecutive to the first pulse pattern, wherein a pulse period defined between a start of the first pulse pattern and a start of the second pulse pattern is in a range from 1 millisecond (ms) to 100 ms; and
delivering the waveform using at least one of a plurality of electrodes on a catheter, wherein the pulse period of the waveform facilitates increasing lesion depth.

16. The method in accordance with claim 15, wherein the pulse period is in a range from 5 ms to 100 ms.

17. The method in accordance with claim 15, wherein the pulse period is in a range from 10 ms to 100 ms.

18. The method in accordance with claim 15, wherein the first and second pulse patterns are included within a burst, and wherein the burst includes one hundred fifty total pulse patterns or less

19. The method in accordance with claim 15, wherein the second pulse pattern is the last pulse pattern in a burst, and wherein the second pulse pattern has a larger applied voltage and/or a longer pulse width than the first pulse pattern.

20. The method in accordance with claim 15, wherein at least one of the first and second pulse patterns is a biphasic pulse pattern.
